(19) United States
(12) Patent Application Publication
(10) Pub. No.: US 2013/0224783 A1
(43) Pub. Date: Aug. 29, 2013

(54) METHOD FOR DETECTION OF LIPOSOLUBLE VITAMINS

(71) Applicants: Florian J. Schweigert, Berlin (DE); Ralf Mothes, Nuthetal (DE)

(72) Inventors: Florian J. Schweigert, Berlin (DE); Ralf Mothes, Nuthetal (DE)

(73) Assignee: BAPaT GmbH, Teltow (DE)

(21) Appl. No.: 13/825,564

(22) PCT Filed: Sep. 21, 2011
(86) PCT No.: PCT/EP2011/066424

§ 371 Date: Apr. 15, 2013

(30) Foreign Application Priority Data
Sep. 22, 2010 (DE) 10 2010 046 912.2

(51) Int. Cl. G01N 33/82

(52) U.S. Cl. CPC G01N 33/82
USPC 435/29; 436/131; 436/20; 435/288.7; 422/430

(57) ABSTRACT

The invention relates to a method for analysis of liposoluble vitamins of biological materials. It is proposed to perform reagent storage, sample application and conducting of the measuring in one reaction vessel and to correct interfering ingredients during the photometric evaluation.

### FIELD OF APPLICATION AND PRIOR ART

Colorimetric methods are scarcely used in current vitamin analytics, since they are hard to implement for different reasons. For instance, the reagent is relatively unstable and a sample needs to be mixed with high temporal accuracy since the reaction may be observed but for a very short time. Furthermore, the working temperature is to be exactly maintained since the color reaction depends on the temperature to a great extent. Moreover, ingredients which can affect the color reaction in an undesired manner can be found in various biological materials which are the basis of the analytics.

The colorimetric detection of liposoluble vitamins, in particular of vitamin A and vitamin D, is based on the reaction of said compounds and a color reagent which in the presence thereof results in a concentration-dependent color reaction. For example, the specific colorimetric detection of vitamin A (in the type of retinol and retinyl esters) is based on the reaction with antimony(III) chloride (SbCl₃), dissolved in chloroform. Alternative color reagents are, for example, trifluoroacetic acid, trichloroacetic acid or other reagents known to the persons skilled in the art or to be used in the future which are based on comparable approaches. For example dichloromethane or hexane can be considered as solvent, besides chloroform. However, also other suitable solvents or solvent mixtures known to the persons skilled in the art can be applied.

For said analytics, so far the reagents had to be newly prepared on a regular basis, be metered by means of specific devices and added to the sample. Due to the high rate and the pronounced temperature dependence of the color reaction, the measuring instruments need to be extensively prepared and the results need to be evaluated subsequently. Furthermore, the samples need to be prepared in advance by means of different methods because of compounds possibly interfering with the color reaction. In the case of fat-containing samples, usually this is a saponification step.

These specific properties make the use of such methods for quantification of liposoluble vitamins extremely difficult, especially under non-laboratory conditions. For this reason, nowadays analyses are in general exclusively performed by means of high-performance liquid chromatography (HPLC). Indeed, this is very exact, but at the same time very labor intensive and costly in terms of instruments. Although detection is needed under simple conditions, such as for example in developing countries, there is no way to perform it.

Thus, there is a high demand for a method for analysis of liposoluble vitamins in biological materials, in particular in oils or fat, which can be conducted in a simple analytic environment, also with unprepared or non-purified samples.

### Object and Solution

It is therefore the object of the present invention to provide a method for colorimetric analysis of liposoluble vitamins in biological materials which improves the stability of the reagents and the control of the time-sensitive and temperature-sensitive color reaction. Furthermore, if possible, a quantitative analytics shall be made possible even in the presence of interfering ingredients. Finally, it is the object of the invention to provide a device for analysis of ingredients obtained by means of the method.

Thus, the aim of the method is to improve the detection to be performed currently only under laboratory conditions by means of a color reaction such that a quantitative laboratory-independent rapid test for liposoluble vitamins, in particular vitamin A and vitamin D, is possible. Such a test is above all also important for the difficult analytic conditions in developing countries and newly-industrialized countries.

The aforementioned objects are achieved by the method having the features of claim 1 as well as by the kit having the features of claim 15. Preferred embodiments of said method and said kit are described in the dependent claims 2 to 14 or 16 and 17. The wording of all claims is hereby incorporated into the content of the present description by reference.

The present invention achieves the underlying technical problem by means of the aforementioned method for analysis of liposoluble vitamins, in particular vitamin A, vitamin D and vitamin E, in biological materials. This concerns the stability of the reagent, the control of the progress of the color reaction as well as the presence of other interfering compounds which in particular also lead to color reactions.

In the method described, the stability of the reagent could be improved from the usually few weeks to up to 2 years. The form of application of the sample to be examined now allows a to-the-second initiation of the time-sensitive color reaction and specific evaluation methods allow measuring of the vitamins even in samples rich in compounds interfering with the color reaction. Another approach allows to standardize the temperature deviations. As a result, the calorimetric method for detection of liposoluble vitamins can be conducted even under less controlled conditions than in a laboratory environment.

The objects were also achieved by a test kit, comprising a reagent vessel in which both storage of the reagent mixtures and application of the sample are effected. At the same time, the reagent vessel preferably also serves as measuring chamber for the color reaction. Measuring is effected preferably in a portable photometer which is adapted to the optic conditions of the measuring container and which allows an evaluation of the results. In this case, interferences by other ingredients of the sample and the temperature differences can be considered and corrected.

Use of all steps or parts of the described method in other constellations is a constituent part of the invention. This relates to the use of other measuring vessels, such as standard photometers, or the use of modified measuring vessels, for example which, in the basic configuration thereof, correspond to the claim according to the invention.

Furthermore, according to the invention this also relates to standard measuring methods and the application of the evaluation method to values as obtained on a standard photometer.

Central aspects of the invention shall now be discussed in the following with reference to the corresponding headings. It is understood that according to the invention the features described in the individual sections can also be realized in combinations thereof.

Improvement of Reagent Stability

Since generally the reagents used in the invention are very sensitive to water (hygroscopic), they are to be subject to corresponding purifying methods and drying methods known to the persons skilled in the art. Subsequent thereto, a storage is possible for a limited time period of a few weeks only.

In the invention, the purified and dried solvents (such as chloroform, dichloromethane, hexane, etc.) and chemicals (antimony(III) chloride, trifluoroacetic acid, trichloroacetic acid etc.) are mixed according to compositions and concentrations known to the persons skilled in the art and filled in glass containers which can be sealed in a gas-tight or air-tight manner. Corresponding to the claim according to the invention, the sealing is preferably effected by means of a cap which contains a septum through which, for example, the sample can be injected subsequent thereto. The sealing can be achieved, for example, by crimping, screwing or adhesion. However, also other methods for gas-tight sealing are provided according to the invention.

The septum can consist of different materials which can fulfill at least two functions, namely stability against the reagents and tightness against evaporation of the solvent and invasion of air and water, for example of water vapor. Another criterion is the possibility of injecting the sample, for example by means of a syringe via a cannula.

Due to its high stability and the high gas tightness, the best option for the vessel is that it is made of glass, however also other known and unknown materials having comparable characteristics regarding stability against organic solvents and etching substances are conceivable.

In the case according to the invention, the vessel preferably not only serves for storage of the reagents but also as a reaction vessel and a measuring chamber which can be placed into a measuring instrument.

Control of the Color Reaction

The color reaction between reagents and analytes depends on time and temperature. Especially in the case of vitamin A, the reaction usually proceeds within a few seconds. Usually, it reaches its maximum after 5 seconds and is completed after 60 seconds. For this reason, the time of a reaction start has to be controlled carefully. Usually, this is achieved by adding the reagent to the sample and by rapid agitation.

Surprisingly it was found that also with an inverse step the adding of the sample to be examined to the reagent is possible, initially without a reaction occurring. In this case, not the reagent is added to the sample, but the sample is added to the reagent. This is particularly effective if reagent and sample differ in terms of density and/or viscosity.

An oil sample or fat sample of vegetable or animal origin can, for example, be layered on the reagent easily due to the significant differences in their density and viscosity in relation to all solvents used.

In the case of other sample compositions, the reagent system used can be optimized, where required. Thus, a hexane extract of the liposoluble vitamins can easily be layered on a reagent containing chloroform or dichloromethane.

According to the invention, overlaying is effected best in a reaction vessel which can also be used for mixing and measuring.

By means of the layering, the sample and the reagent are in direct contact without the color reaction being initiated. In general, the interfacial reaction usually occurring at the interphase can be neglected.

According to the invention, the two phases are then mixed by means of a brief agitation of the two phases and the color reaction is initiated.

According to the invention, in terms of their viscosity, the phases differ from one another preferably at least by the factor 2, better at least by the factor 5 to 50, and ideally by more than the factor 100. Thus, oil having a viscosity of approximately 10² (η in mPa*s) differs from the commonly used solvents hexane (0.320) and chloroform (0.560) by more than the hundredfold.

Furthermore, according to the invention, in terms of their density, the phases differ from one another by more than 5%, better by more than 10 to 50%, and even better by at least 100%. When using oil (0.8-0.9 g/cm³) and n-hexane (0.66 g/cm³) as a sample or carrier of the sample, a clear phase separation can be achieved using chloroform (1.48 g/cm³) or dichloromethane (1.33 g/cm³) .

Control of Temperature

The color reaction with liposoluble vitamins exhibits a significant temperature dependency, in particular in the case of vitamin A.

According to the invention, said influencing variable can be considered in an adjusting manner by the simultaneous measuring of the temperature within or in the proximity of the measuring cell of the measuring instrument.

Furthermore, the measuring accuracy can particularly be improved in the lower region by means of control of the temperature.

In the method according to the invention, the color reagents are preferably cooled down and the sample is usually added, for example by injection through a septum, at room temperature or is preferably also cooled down.

According to the invention, tempering is effected between +40° C. and −80° C. Preferably, the temperature range is between +30° C. and −18° C., even more preferred in the range between 20° C. and 4° C.

Depending on air humidity, after the cooling of the reaction vessel, in particular of test tubes, and measuring at ambient temperature condensed water can form on the outer face of the tube. In order to prevent the formation of condensed water on the tube outer face, the tubes can be subject to a so-called anti-fog coating or anti-fog treatment prior to or subsequent to the cooling or already during the production process. In the simplest case, the vessel can simply be wiped-off.

In the context of the invention, an anti-fog coating or an anti-fog treatment refers to a special surface treatment on crystal-clear surfaces, which prevents a fogging (condensing) under influence of water vapor.

Thus, by means of special sprays or liquids, so-called surfactants, known to the persons skilled in the art, it can be prevented that microscopic droplets are formed during the condensation of water vapor which scatter the light and which let the clear parts of the surface become almost completely or completely opaque.

An alternative method is, for example, the coating with silicon nanoparticles embedded in a polymer film. The application is effected by means of a method known to the persons skilled in the art.

Consideration of Interfering Substances

In particular biological materials contain substances and ingredients which can have an interfering effect to the color detection of liposoluble vitamins. Usually these are substances including multiple double bonds. The latter can either affect the reaction kinetics or interfere with the measurement by a distinct color reaction. If the color reaction takes place in a wave length range different from the vitamin-typical reaction, then the interference can be considered by means of measuring techniques and recording of different wave lengths with a wave length difference measurement.

According to the invention, the measuring is effected in a wave length range between 250 and 900 nm, better at 370-800 nm and best at 450-700 nm.

If the color reaction of the interfering substances is in the wave length range of the color reaction of the vitamin, especially in the range of 620 nm in the case of the color reaction of vitamin A, the aforementioned correction is not possible. In this case, assignments can be achieved by means of differences in the time reaction of the kinetics of the different components and thereby the analyte can be quantified.

For example, ingredients of palm oil react with the color reagent for vitamin A in a comparable wave length range as vitamin A per se. Unlike the case of the color reaction for vitamin A, said color reaction is not short and fading rapidly, but stable over the time period of the color reaction of vitamin A. According to the invention, the calculation is either effected via the rate of the decline of the reaction or by means of the difference measured at two points of time, for example.

According to the invention, the course of time between 1 and 2000 seconds is measured. Better between 3 and 600 seconds and even better between 3 and 300 seconds. At best between 3 and 60 seconds.

In the context of the present invention, “biological materials” are meant to refer to materials derived from vegetables, animals, humans and/or microorganisms, in particular materials produced naturally in the body, preferably samples of materials produced naturally in the body.

In the context of the present invention, “microorganisms” shall comprise besides eukaryotes, such as algae, prokaryotes and/or funguses, such as yeasts, for example, even viruses. In particular, “biological materials” shall comprise culture-derived organisms as well as culture supernatants.

The method according to the present invention is provided for analysis of liposoluble vitamins, preferably of vitamin A, vitamin D and vitamin E of biological materials.

Analysis of the biological materials is effected directly, preferably with a sufficient liquidation and with a water content of less than 5%, in particular between 2 and 4%, or preferably between 0.01 and 2%. In the case of samples having a higher water content, generally a pretreatment in the form of an extraction of the ingredients to be analyzed is required. Each method known to the person skilled in the art can be used in this case.

In a preferred embodiment of the method, food of animal and/or vegetable origin, in particular homogenates of food, are used as biological materials.

As food, in particular oils or fat of animal or vegetable origin can be considered. Moreover, by means of the method according to the invention, milk and dairy products such as butter, eggs, preferably full egg yolk, fruits and vegetables may be analyzed.

Preferably, food containing liposoluble vitamins comes into consideration for the method according to the invention.

In another preferred embodiment of the method, tissues, organs and/or body fluids are used as biological materials.

In another preferred embodiment, such biological materials are used which are applied for diagnostics and/or therapy monitoring in the medical field.

In another preferred embodiment of the method, blood, plasma, serum or milk are used as body liquids and/or secretion.

In another embodiment of the invention, the method is conducted at a pressure between 0.5 bar and 5 bar, in particular between 0.8 bar and 2 bar.

Preferably, the biological materials are placed into a solvent-resistant environment prior to the initiation of the color reaction with the reagents.

In another preferred embodiment, a vessel made of glass is used as solvent-resistant environment.

Advantageously, in the method according to the invention, vessels can be used which seal the content, i.e. generally the color reagent dissolved in a solvent, in an air-tight manner. The air-tight sealing prevents the access of humidity and thus improves the stability of the reagent or of the reagents.

Preferably vessels are used which are configured such that they are suitable for spectroscopic examinations.

Preferably, there are vessels used, wherein the sample can be transferred into the vessel through a septum by means of an applicator, preferably by means of a syringe with a cannula.

For the subsequent analysis of the ingredients, all known or even thus far unknown analysis techniques can be considered.

Furthermore, the present invention solves the underlying technical problem also by means of the provision of a spectrophotometer, in particular of a hand photometer, for measuring of ingredients of biological materials in a cylindrical vessel, wherein the optical path of the photometer is adjusted such that the measuring detects an upper half of the vessel.

In the scope of the present invention, a cylindrical vessel refers to a vessel that has a constant profile essentially over the entire length thereof. As a constant profile, for example a rectangular cross-section, in particular a square cross-section as used in spectroscopic cuvettes, can be considered. Advantageously, for the measuring of the ingredients of the biological materials, those vessels are used in which the method according to the invention was conducted. Advantageously, an analysis of the ingredients is effected without time delay in the spectrophotometer.

Summing up, it has to be noted that the present invention significantly improves the quantitative colorimetric detection of liposoluble vitamins in terms of the precision thereof and significantly simplifies it in terms of the handling thereof. Furthermore, the exposure of the examiner to chemicals potentially hazardous to health is significantly reduced. In particular the analytics of oils and fat are simplified thereby. Furthermore, extensive preparation methods, such as saponification of the samples, are not required. The invention can be conducted in one step. It is therefore particularly suitable for miniaturized methods, in particular high throughput methods known in the type of chip laboratories and used preferably in so-called “point-of-care” systems.

Further features and advantages of the invention result from the subsequent description of preferred embodiments with reference to examples in connection with the dependent claims. In this case, the individual features can in each case be realized on their own or in a combination of multiple features thereof.

### DETAILED DESCRIPTION OF EXEMPLIFIED EMBODIMENTS

In the following, the invention is further described and explained with reference to selected exemplary embodiments in connection with the attached drawings: In the drawings:

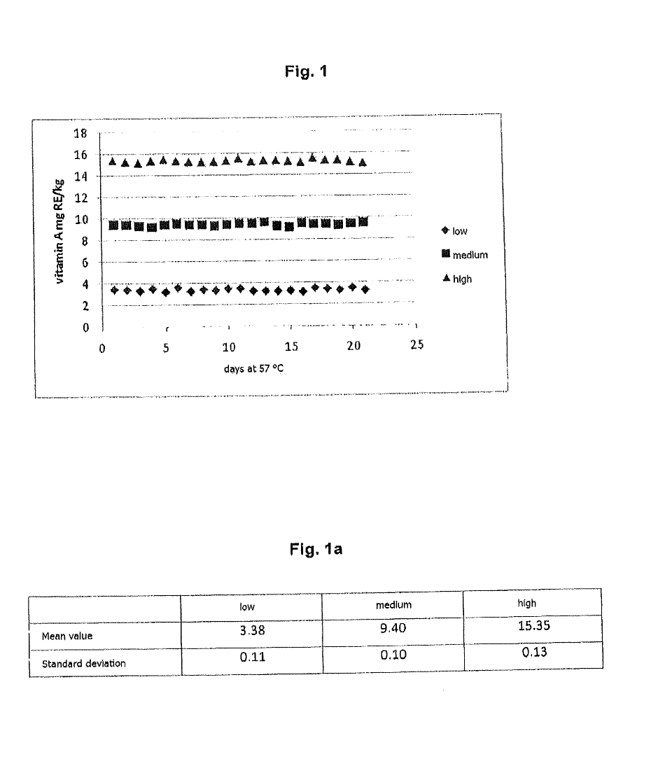

FIG. 1 shows the stability of the reagent under stress test conditions (storage at 57° C. for 3 weeks);

FIG. 1a shows a tabular comparison of the FIG. 1 shown as mean value and standard deviation.

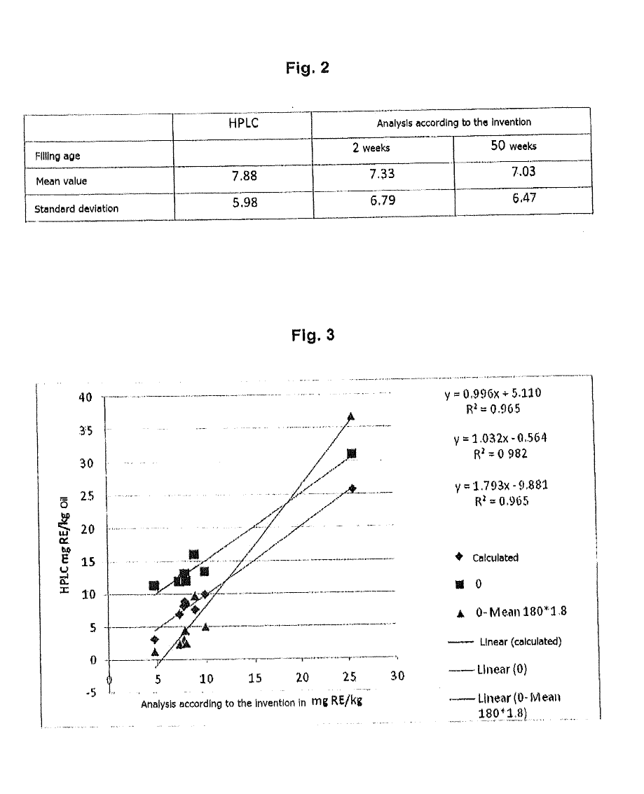

FIG. 2 shows a tabular comparison of measuring results of an analysis according to the invention in test tubes which were newly filled and such ones stored for 50 weeks at room temperature (18-29° C.) under conditions according to the present invention.

FIG. 3 shows the results of the evaluation of palm oil samples enriched with different contents of vitamin A prior to and after the data correction according to the invention in the portable photometer. The values are compared in relation to the HPLC measuring.

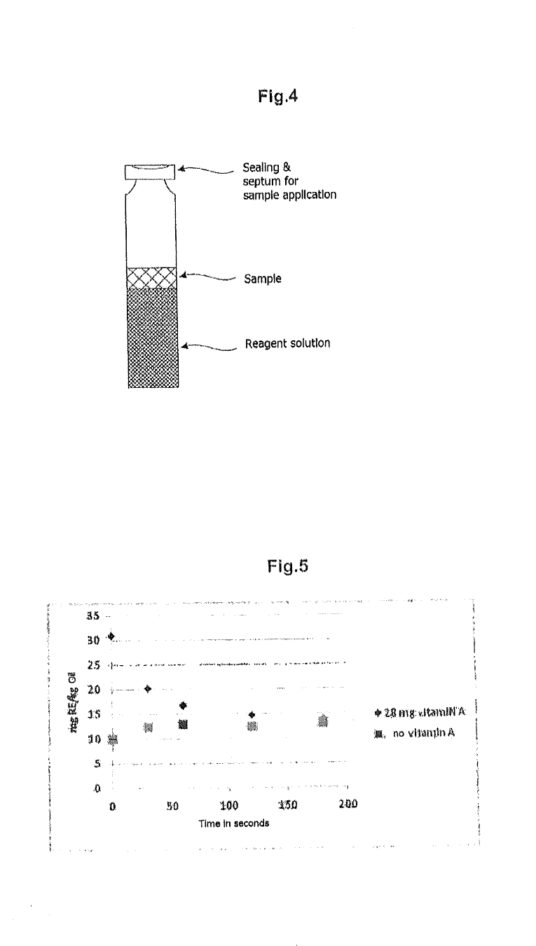

FIG. 4 shows a graphical illustration of the over-layering of the analysis reagent with an oil sample under the conditions according to the invention.

FIG. 5 shows the kinetics of the color reaction of a palm oil sample with and without vitamin A enrichment.

FIG. 6 shows the influence of temperature to the intensity and stability of the color reaction.

FIG. shows the linearity of the measuring of vitamin D in oil samples of different concentrations.

FIG. 1 shows a diagram where the storage stability of a reagent filled-up and stored under conditions according to the invention was tested under so-called stress conditions at 57° C. This period of time corresponds to a storage at room temperature over at least 2 years.

What was tested was the stability with three palm oil samples differing in terms of their vitamin A content. Measuring was effected according to the invention.

The results show that the measurements are stable over the entire time period.

FIG. 1a shows the values of FIG. 1 as mean value and standard deviation. The results show the comparability in both values.

FIG. 2 shows an illustration where the measuring results of vitamin A in palm oil from 14 samples with different concentrations are shown. The reagents were filled-up and stored as described by the invention. Storage was at room temperature (18-29° C.) for 2 and 50 weeks, respectively. FIG. 2 shows the results of the illustration as mean value and standard deviation. The results show the comparability in both values.

The palm oil samples were determined according to FIG. 3 initially without correction using the method according to the invention (described as 0). Subsequent thereto, the average mean value of the basic absorption after 180 seconds was subtracted from the measured values (0 values). In another step according to the invention, a calculation of the values based on kinetics (time progress) over 30 seconds was performed. The results (shown as calculated) show that by means of this method, the best-possible conformance to the values detected via the HPLC as golden standard can be achieved.

According to FIG. 4, the liquid or liquefied sample is applied according to the invention via the septum located in the sealing cap. In this case, a careful over-layering of the reagent with the sample is effected. Two phases form as a result. The initiation of the reaction is triggered not earlier than by means of the mixing inside the reaction vessel.

Two oil samples with and without vitamin A corresponding to the analysis according to the invention show a different reaction progress as shown in FIG. 5. The results of the analysis according to the invention show the interfering influence of undesired ingredients in oil.

FIG. 6 shows the conducting of the analysis according to the invention under different temperature conditions and their effect on temporal characteristics at 20° C., 30° C. and 40° C.

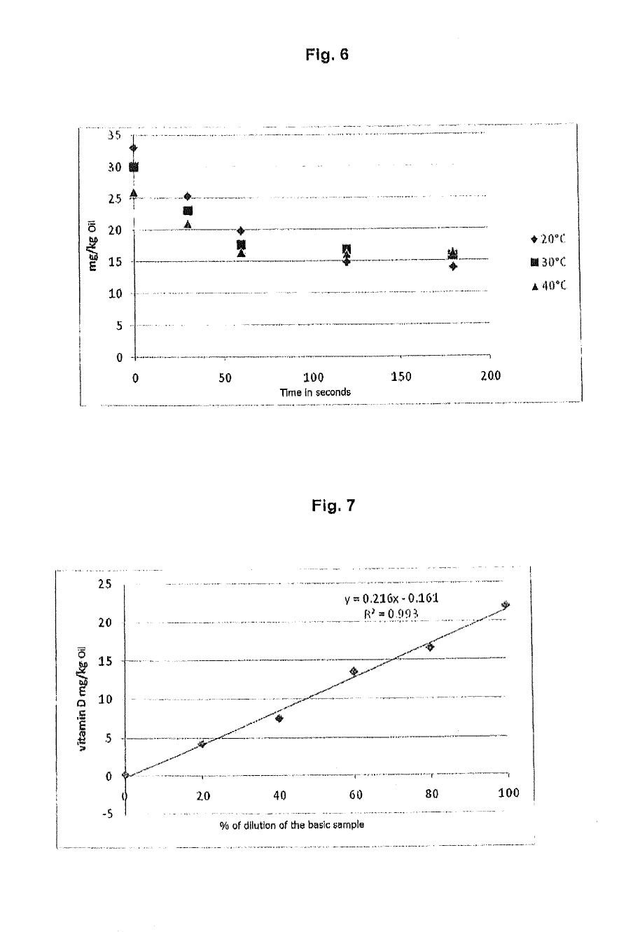

The analysis of samples containing vitamin D was conducted according to the invention. Measuring was effected in the spectrophotometer. The oil sample enriched with vitamin D was diluted in five equidistant distances and subsequently measured according to the invention. This is shown in FIG. 7.

Exemplary Embodiments

Detection of Vitamin A from Vegetable Oils

The oil was introduced into the reaction vessel by means of an applicator (syringe with needle) in a predefined volume of 500 μl. For this purpose, the sealing septum of the vessel was penetrated with the cannula and the oil was slowly layered over the reagent already present in the reagent vessel. Therein, no visible mixtures occurred and two clearly delimited phases were formed. The oil sample as an upper phase, the reagent therebelow. By means of the phase separation, an early and uncontrolled color reaction was prevented. The color reaction was subsequently started by means of a short, vigorous agitation of the reagent vessel. Depending on the vitamin A concentration in the sample, a more intensive coloration was obtained. The color intensities are measured directly in the spectral photometer.

Extraction and Evaluation of Vitamin D in Oils

The experimental procedure was maintained as described above. Likewise the measuring of the sample. The color intensity was directly proportional to the concentration of vitamin D in the sample (FIG. 7). The analysis according to the invention was tested in terms of linearity of the enriched oil samples. Linearity was 0.956. The correctness of the results was validated by means of the golden standard HPLC.

Extraction and Evaluation of Vitamin A and Vitamin D from Margarine

Margarine was used as biological material. The experimental procedure and the measuring were conducted as described above. However, prior to the measuring, an extraction of the lipids into organic solvents was effected. This was performed in a manner known for example from a method described in DE 10 2006 044 795 A1. The margarine sample enriched with vitamin A and vitamin D was initially slightly heated in order to liquefy it. Subsequently, the sample was transferred into hexane. These methods are well-known to the persons skilled in the art. After the extraction of the sample in hexane, the extract was transferred to the reagent using the aforementioned method according to the invention and correspondingly layered there onto. The color reaction was initiated by mixing of the sample and the reagent. The color characteristics had two phases. Initially, a blue coloration occurred, then the vitamin-D-typical color reaction of pink-red. The intensity of the color reaction for vitamin A and vitamin D corresponded to the results determined by means of HPLC.

1. A method for preferably quantitative colorimetric determination of liposoluble vitamins in biological materials, in particular of vitamin A, vitamin D and/or vitamin E, where the type of filling of the reagent or the reagents and the application of the sample result in a prolongation of the reagent stability and the controlled start of the time and temperature critical color reaction, and in particular by omission of a pretreatment, the interfering influences of foreign substances are corrected by means of special measuring and analysis methods in a photometer.

2. The method according to claim 1, characterized in that reagent storage, sample application, and performing of the color reaction take place in one reaction vessel.

3. The method according to claim 1, characterized in that the sample application to the analysis reagent is effected such that there is a clear phase separation between reagent and sample with formation of at least two phases, wherein preferably the color reaction is triggered by the mixing of the two phases.

4. The method according to claim 1, characterized in that the temperature of the reagent is maintained constant for improvement of the automatic quantification.

5. The method according to claim 1, characterized in that the measuring methods are one or more different spectroscopic measuring methods at different wave lengths.

6. The method according to claim 1, characterized in that for evaluation a difference between different color reactions is used.

7. The method according to claim 1, characterized in that the decline rate of the vitamin-specific color reaction is used for the quantification.

8. The method according to claim 1, characterized in that the ingredients are examined during a time period of 3 seconds to 10 minutes, in particular of 10 seconds to 5 minutes, preferably of 5 seconds to 2 minutes.

9. The method according to claim 1, characterized in that the biological materials comprise vegetable, animal, human and/or microbial materials which originate in particular from cell culturing.

10. The method according to claim 1, characterized in that the biological materials are subject to pretreatments prior to the use thereof, in particular to purification processes.

11. The method according to claim 1, characterized in that as biological materials, food of animal and/or vegetable origin, in particular homogenates of food, are used, wherein preferably oils and fat of vegetable or animal origin are used as food.

12. The method according to claim 1, characterized in that tissues, organs and/or body fluids are used as biological materials.

13. The method according to claim 1, characterized in that the method is conducted at a temperature in the range of −80° C. to 60° C., in particular in the range of 8° C. to 30° C.

14. The method according to claim 1, characterized in that the method is conducted at a pressure between 0.5 bar to 5 bar, in particular between 0.8 bar to 2 bar.

15. A kit for the preferably quantitative colorimetric detection of liposoluble vitamins in biological materials, in particular of vitamin A, vitamin D and/or vitamin E, comprising at least one vessel which contains the reagents provided for the colorimetric detection and which is sealed against the environment in an air-tight manner, wherein the vessel is preferably configured for direct use in a photometer, in particular a portable photometer.

16. The kit according to claim 15, characterized in that the vessel comprises at least one septum for introducing of a sample containing the liposoluble vitamins.

17. The kit according to claim 15, characterized in that the vessel is provided with a so-called anti-fog coating on at least a part of the outer surface thereof.
